(19) United States
(12) Patent Application Publication
(10) Pub. No.: US 2014/0343008 A1
(43) Pub. Date: Nov. 20, 2014

(54) HEPATITIS C TREATMENT

(71) Applicant: Gilead Pharmasset LLC, Foster City, CA (US)

(72) Inventor: Cheng Yong Yang, Foster City, CA (US)

(21) Appl. No.: 14/169,004

(22) Filed: Jan. 30, 2014

Related U.S. Application Data
(60) Provisional application No. 61/919,108, filed on Dec. 20, 2013.
(60) Provisional application No. 61/824,266, filed on May 16, 2013.

(51) Int. Cl. A61K 31/7072; A61K 31/501; A61K 31/381; A61K 31/5377; A61K 31/4188; A61K 31/4184

(52) U.S. Cl. CPC A61K 31/7072; A61K 31/4188; A61K 31/4184; A61K 31/381; A61K 31/5377; A61K 31/501
USPC 514/51

(57) ABSTRACT

Disclosed are methods for treating hepatitis C in a human patient in need thereof that entails administering to the patient of about 350 mg of sofosbuvir and another anti-HCV compound.

### CROSS REFERENCE TO RELATED APPLICATIONS

This application claims the benefit under 35 U.S.C. §119(e) to U.S. Provisional Application Ser. No. 61/824,266, filed on May 16, 2013, and U.S. Provisional Application Ser. No. 61/919,108, filed on Dec. 20, 2013, the entireties of which are incorporated herein by reference.

### BACKGROUND

Hepatitis C is recognized as a chronic viral disease of the liver which is characterized by liver disease. Although drugs targeting the liver are in wide use and have shown effectiveness, toxicity and other side effects have limited their usefulness. Inhibitors of hepatitis C virus (HCV) are useful to limit the establishment and progression of infection by HCV as well as in diagnostic assays for HCV.

Sofosbuvir (SOF), having a chemical name of (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate, is a selective inhibitor of non-structural protein 5B (NSSB). Sofosbuvir is described in, for example, WO 2010/132601 and U.S. Pat. No. 7,964,580.

The compound, methyl {(2S)-1-[(2S,5S)-2-(9-{2-[(2S,4S)-1-{(2R)-2-[(methoxycarbonyl)amino]-2-phenylacetyl}-4-(methoxymethyl)pyrrolidin-2-yl]-1H-imidazol-5-yl}-1,11-dihydroisochromeno[4′,3′:6,7]naphtho[1,2-d]imidazol-2-yl)-5-methylpyrrolidin-1-yl]-3-methyl-1-oxobutan-2-yl}carbamate, designated herein as “Compound I”, is a selective inhibitor of non-structural protein 5A (NSSA). Compound I is described in, for example, WO 2013/075029.

The compound, methyl ((S)-1-((1R,3S,4S)-3-(6-(9,9-difluoro-7-(2-((S)-5-((methoxycarbonyl)-L-valyl)-5-azaspiro[2.4]heptan-6-yl)-1H-imidazol-5-yl)-9H-fluoren-2-yl)-1H-benzo[d]imidazol-2-yl)-2-azabicyclo[2.2.1]heptan-2-yl)-3-methyl-1-oxobutan-2-yl)carbamate, designated herein as “Compound II”, is a selective inhibitor of NSSA. Compound II is described in, for example, U.S. Pat. No. 8,088,368.

The compound, 5-(3,3-dimethylbutyn-1-yl)-3-[(cis-4-hydroxy-4-{[(3S)-tetrahydrofuran-3-yloxy]methyl}cyclohexyl) {[(1R)-4-methylcyclohex-3-en-1-yl]carbonyl}amino]thiophene-2-carboxylic acid, designated herein as “Compound III”, is a selective inhibitor of non-structural protein 5B (NS5B). Compound III is described in, for example, U.S. Pat. No. 8,513,298.

The compound, (1R,2R)-1-((2S,4R)-1-((2S)-2-(((((1R,5S)-bicyclo[3.1.0]hexan-3-yl)oxy)carbonyl)amino)-3,3-dimethylbutanoyl)-4-((8-chloro-2-(2-(isopropylamino)thiazol-4-yl)-7-(2-morpholinoethoxy)quinolin-4-yl)oxy)pyrrolidine-2-carboxamido)-2-ethylcyclopropane-1-carboxylic acid, designated herein as “Compound IV”, is a selective inhibitor of non-structural protein 3 (NS3). Compound IV is described in, for example, U.S. Pat. No. 8,178,491.

The compound, 5-((6-(2,4-bis(trifluoromethyl)phenyl)pyridazin-3-yl)methyl)-2-(2-fluorophenyl)-5H-imidazo[4,5-c]pyridine, designated herein as “Compound V”, is a selective inhibitor of non-structural protein 5B (NS5B). Compound V, also known as tegobuvir, is a known compound.

### SUMMARY

Compound I, methyl {(2S)-1-[(2S,5S)-2-(9-{2-[(2S,4S)-1-{(2R)-2-[(methoxycarbonyl)amino]-2-phenylacetyl}-4-(methoxymethyl)pyrrolidin-2-yl]-1H-imidazol-5-yl}-1,11-dihydroisochromeno[4′,3′:6,7]naphtho[1,2-d]imidazol-2-yl)-5-methylpyrrolidin-1-yl]-3-methyl-1-oxobutan-2-yl}carbamate, has the following chemical formula:

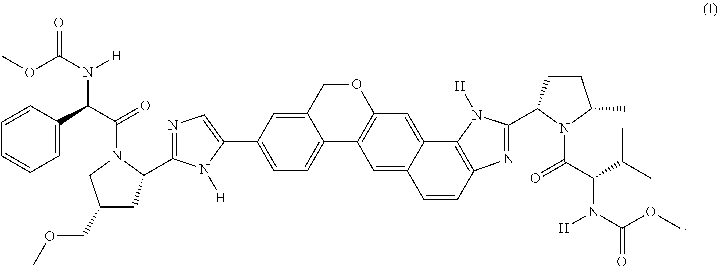

Compound II, methyl ((S)-1-((1R,3S,4S)-3-(6-(9,9-difluoro-7-(2-((S)-5-((methoxycarbonyl)-L-valyl)-5-azaspiro[2.4]heptan-6-yl)-1H-imidazol-5-yl)-9H-fluoren-2-yl)-1H-benzo[d]imidazol-2-yl)-2-azabicyclo[2.2.1]heptan-2-yl)-3-methyl-1-oxobutan-2-yl)carbamate, has the following chemical formula:

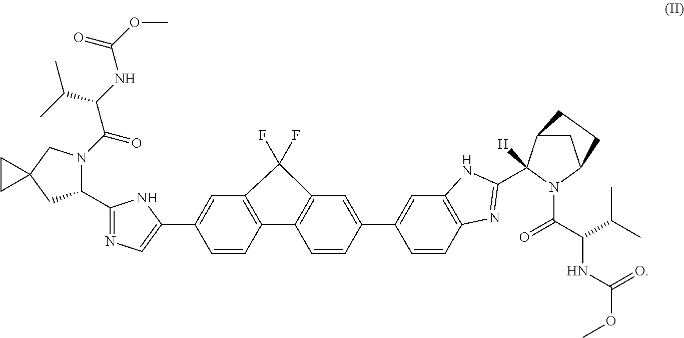 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]

Compound III, 5-(3,3-dimethylbutyn-1-yl)-3-[(cis-4-hydroxy-4-{[(3S)-tetrahydrofuran-3-yloxy]methyl}cyclohexyl) {[(1R)-4-methylcyclohex-3-en-1-yl]carbonyl}amino]thiophene-2-carboxylic acid, has the following chemical formula:

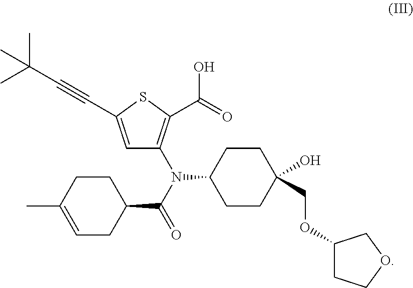 [SMILES: CC1=CC[C@H](C(=O)N(c2cc(C#CC(C)(C)C)sc2C(=O)O)[C@H]2CC[C@](O)(CO[C@H]3CCOC3)CC2)CC1]

Compound IV, (1R,2R)-1-((2S,4R)-1-((2S)-2-(((((1R,5S)-bicyclo[3.1.0]hexan-3-yl)oxy)carbonyl)amino)-3,3-dimethylbutanoyl)-4-((8-chloro-2-(2-(isopropylamino)thiazol-4-yl)-7-(2-morpholinoethoxy)quinolin-4-yl)oxy)pyrrolidine-2-carboxamido)-2-ethylcyclopropane-1-carboxylic acid, has the following chemical formula:

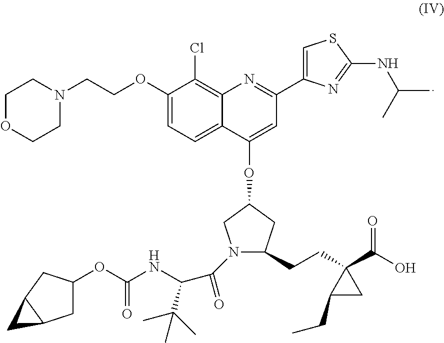 [SMILES: CC[C@@H]1C[C@]1(CC[C@@H]1C[C@@H](Oc2cc(-c3csc(NC(C)C)n3)nc3c(Cl)c(OCCN4CCOCC4)ccc23)CN1C(=O)[C@@H](NC(=O)OC1C[C@@H]2C[C@@H]2C1)C(C)(C)C)C(=O)O]

Compound V, 5-((6-(2,4-bis(trifluoromethyl)phenyl)pyridazin-3-yl)methyl)-2-(2-fluorophenyl)-5H-imidazo[4,5-c]pyridine, has the following chemical formula:

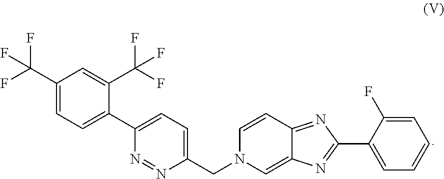 [SMILES: Fc1ccccc1-c1nc2ccn(Cc3ccc(-c4ccc(C(F)(F)F)cc4C(F)(F)F)nn3)cc-2n1]

Sofosbuvir, (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate, has the chemical formula:

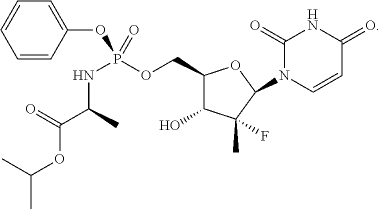 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

It is discovered that when administered along with one or more of the compounds, Compound I-V, sofosbuvir exhibits increased exposure in vivo. It is further shown that Compounds I-V increased the absorption of sofosbuvir by inhibiting the efflux of sofosbuvir. It is further shown that Compounds I-V inhibit the P-glycoprotein 1 (also known as the permeability glycoprotein or P-gp) transporter. As sofosbuvir is a substrate for P-gp, the increased exposure of sofosbuvir in the co-administration may be attributed to the inhibition of P-gp by one or more of the compounds, Compounds I-V.

It is therefore contemplated that, when sofosbuvir is co-administered with one or more of the compounds, Compound I-V, to treat a disease or condition, the dose needed for sofosbuvir may be reduced, as compared to when sofosbuvir is administered alone, without any of the Compounds I-V.

One embodiment of the present disclosure provides a method for treating hepatitis C in a patient, the method comprising administering to the patient an effective amount of a compound selected from the group consisting of Compound I, Compound II, Compound III, Compound IV, and Compound V having the formulae:

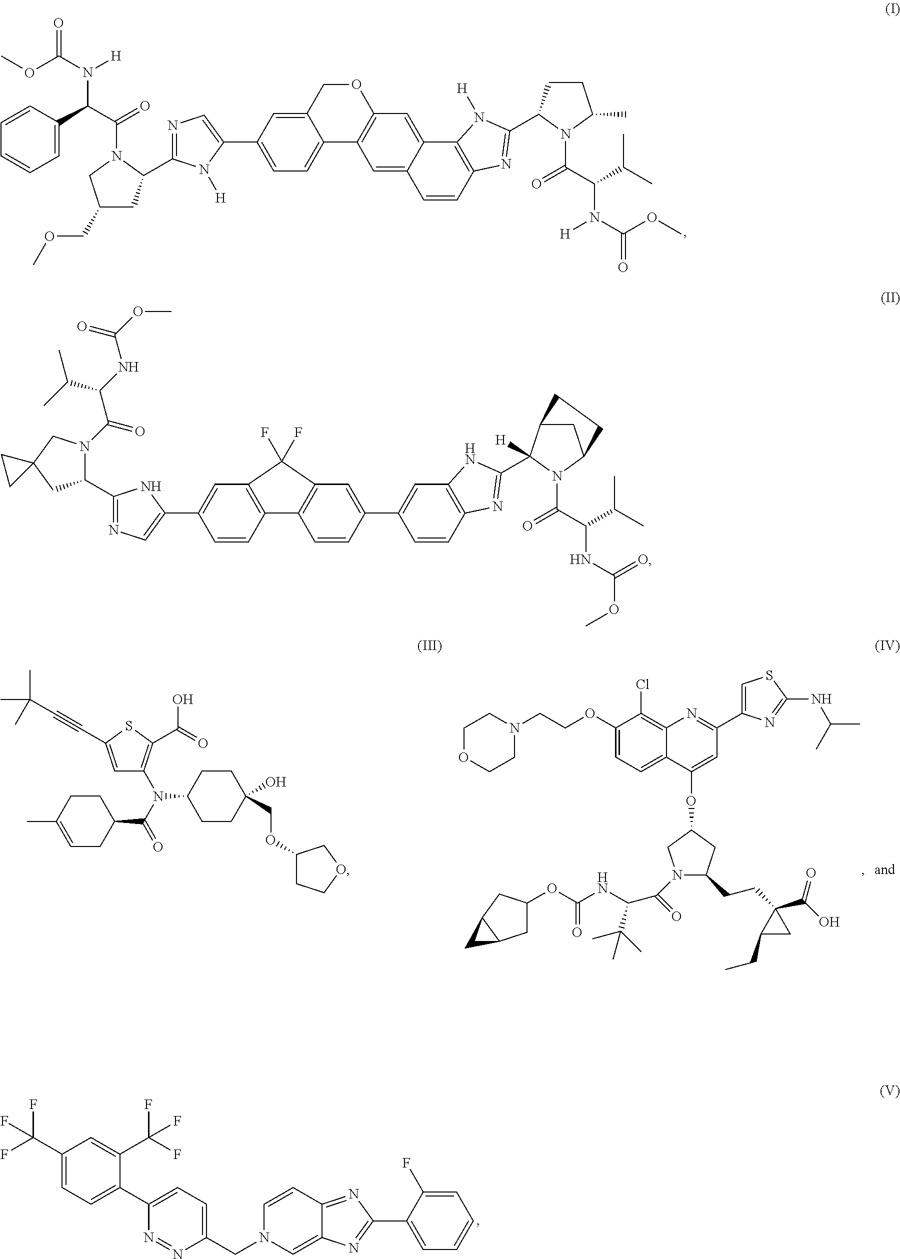

or a combination thereof, and an effective amount of sofosbuvir having the formula

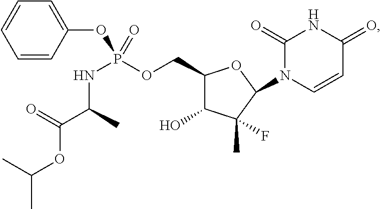 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein the amount of sofosbuvir is about 350 mg daily or less.

In some aspects, the sofosbuvir administered is less than 400 mg daily. In some aspects, the sofosbuvir administered is less than about 390 mg, 380 mg, 370 mg, 360 mg, 350 mg, 340 mg, 330 mg, 320 mg, 310 mg, 300 mg or 250 mg per daily. In some aspects, the sofosbuvir administered is about 350 mg, 300 mg, or 250 mg daily.

In some aspects, the administration is for a period that is not longer than about 8 weeks. In some aspects, the administration is for a period that is not longer than about 7 weeks, 6 weeks, 5 weeks, 4 weeks, or 3 weeks. In some aspects, the administration is for a period of about 6 weeks, 5 weeks, or 4 weeks.

One embodiment of the present disclosure provides a pharmaceutical composition comprising a compound selected from the group consisting of Compound I, Compound II, Compound III, Compound IV, and Compound V having the formulae:

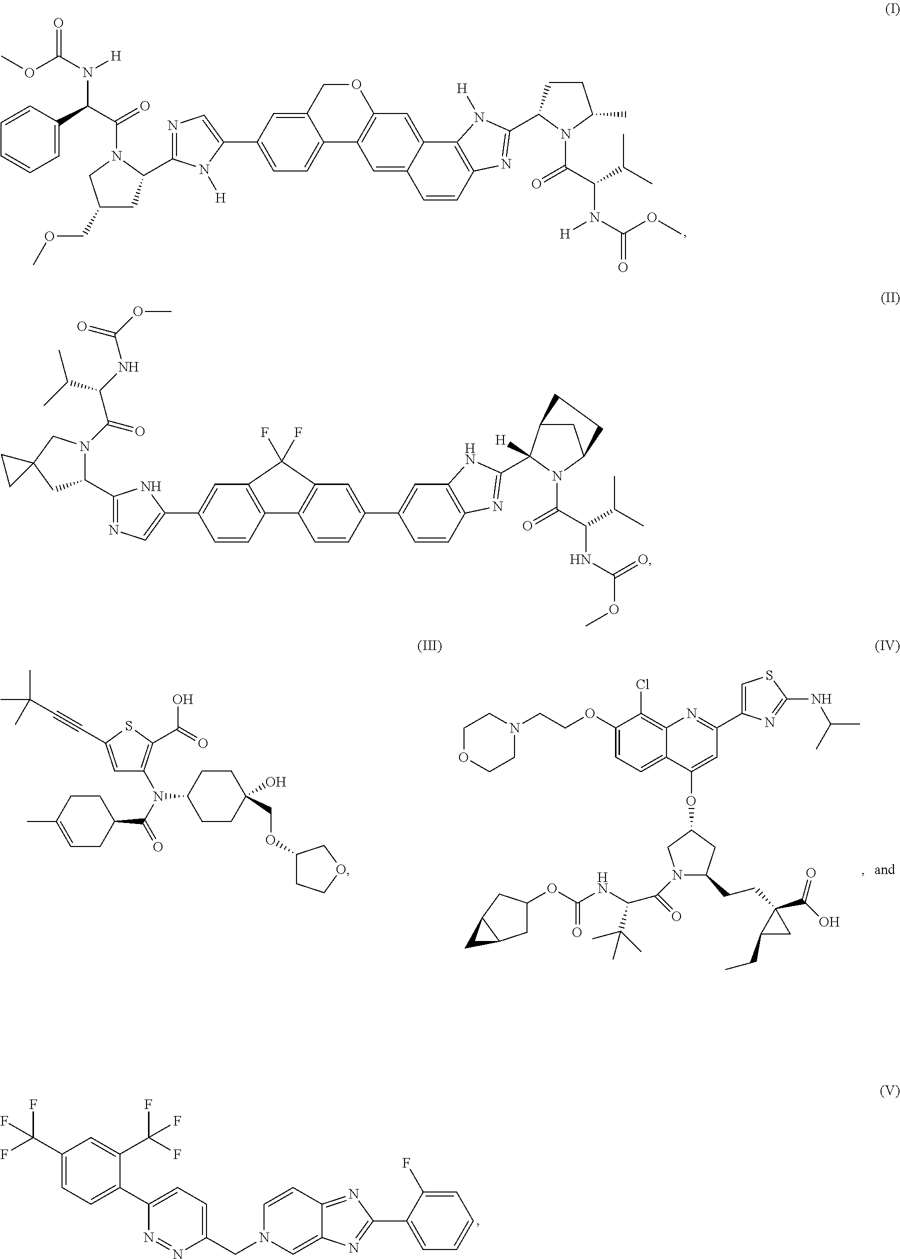

or a combination thereof, and an effective amount of sofosbuvir having the formula:

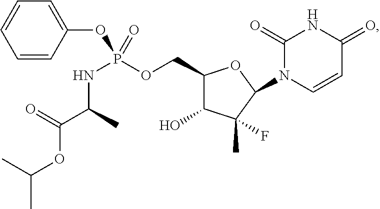 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

wherein the amount of sofosbuvir is about 350 mg or less.

### DETAILED DESCRIPTION

### 1. Definitions

As used in the present specification, the following words and phrases are generally intended to have the meanings as set forth below, except to the extent that the context in which they are used indicates otherwise.

As used herein, the term “about” used in the context of quantitative measurements means the indicated amount ±10%, or alternatively the indicated amount ±5% or ±1%. For example, for a range of ±10%, “about 2:8” would mean 1.8-2.2:7.2-8.8.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectable.

The term “carrier” refers to a glidant, diluent, adjuvant, excipient, or vehicle with which the compound is administered. Examples of carriers are described herein and also in “Remington's Pharmaceutical Sciences” by E. W. Martin.

The term “effective amount” refers to an amount that is sufficient to effect treatment, as defined below, when administered to a mammal in need of such treatment. The therapeutically effective amount will vary depending upon the subject being treated, the weight and age of the subject, the severity of the disease condition, the manner of administration and the like, which can readily be determined by one of ordinary skill in the art.

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “sustained virologic response” refers to the absence of detectable RNA (or wherein the RNA is below the limit of detection) of a virus (i.e. HCV) in a patient sample (i.e. blood sample) for a specific period of time after discontinuation of a treatment. For example, a SVR at 4 weeks indicates that RNA was not detected or was below the limit of detection in the patient at 4 weeks after discontinuing HCV therapy.

The term “% w/w” as used herein refers to the weight of a component based on the total weight of a composition comprising the component. For example, if component A is present in an amount of 50% w/w in a 100 mg composition, component A is present in an amount of 50 mg.

### 2. Combination Therapy

Sofosbuvir (SOF) is a selective inhibitor of non-structural protein 5B (NSSB). Compound I is a selective inhibitor of non-structural protein 5A (NSSA). Compound II is a selective inhibitor of NSSA. Compound III is a selective inhibitor of non-structural protein 5B (NSSB). Compound IV is a selective inhibitor of non-structural protein 3 (NS3). Compound V is a selective inhibitor of non-structural protein 5B (NSSB). It is discovered that Compounds I-V inhibit the P-glycoprotein 1 (also known as the permeability glycoprotein or P-gp) transporter. The present disclosure shows that when administered along with one or more of the compounds, Compounds I-V, sofosbuvir exhibits increased exposure in vivo. Such increased exposure, therefore, may be attributed to the inhibition of P-gp by Compounds I-V, as sofosbuvir is a substrate for P-gp.

One embodiment of the present disclosure provides a method for treating hepatitis C in a patient, the method comprising administering to the patient an effective amount of sofosbuvir and an effective amount of a compound selected from the group consisting of Compound I, Compound II, Compound III, Compound IV, and Compound V or a combination thereof. In some aspects, the sofosbuvir administered is less than 400 mg daily. In some aspects, the sofosbuvir administered is about 350 mg, 300 mg, or 250 mg daily.

In some aspects, the amount of sofosbuvir administered is less than about 390 mg, 380 mg, 370 mg, 360 mg, 350 mg, 340 mg, 330 mg, 320 mg, 310 mg, 300 mg, 290 mg, 280 mg, 270 mg, 260 mg, 250 mg, 240 mg, 230 mg, 220 mg, 210 mg or 200 mg daily. In some aspects, the amount of sofosbuvir administered is from about 100 mg to about 390 mg, or about 150 mg to about 380 mg, or about 200 mg to about 380 mg, or about 200 mg to about 350 mg, or about 250 mg to about 350 mg, or about 200 mg to about 300 mg, or about 250 mg to about 300 mg.

In some aspects, the amount of any of Compounds I-V administered is about 90 mg daily. In some aspects, the amount of any of Compounds I-V administered is about 80 mg or about 100 mg daily. In some aspects, the amount of any of Compounds I-V administered is from about 25 mg to about 200 mg daily, from about 60 mg to about 150 mg daily, from about 70 mg to about 130 mg daily, from about 80 mg to about 110 mg daily, or from about 80 to about 100 daily. In some aspects, the amount of any of Compounds I-V administered is about 30 mg daily.

In some aspects, the administration is for a period that is not longer than about 8 weeks. In some aspects, the administration is for a period of about 6 weeks, 5 weeks, or 4 weeks. In some aspects, the administration is for a period of about 7 weeks, 6 weeks, 5 weeks, 4 weeks, 3 weeks, 2 weeks or 1 week. In some aspects, the administration is for a period that is from about 2 weeks to about 7 weeks, from about 3 weeks to about 6 weeks, from about 3 weeks to about 5 weeks, from about 3 weeks to about 4 weeks, from about 4 weeks to about 6 weeks, from about 4 weeks to about 5 weeks, or from about 5 weeks to about 6 weeks.

In one embodiment, Compounds I-V and sofosbuvir, and pharmaceutical compositions thereof, as described herein are effective in treating one or more of genotype 1 HCV infected patients, genotype 2 HCV infected patients, genotype 3 HCV infected patients, genotype 4 HCV infected patients, genotype 5 HCV infected patients, and/or genotype 6 HCV infected patients. In one embodiment, Compounds I-V and sofosbuvir, and pharmaceutical compositions thereof, as described herein are effective in treating genotype 1 HCV infected patients, including genotype 1a and/or genotype 1b. In another embodiment, Compounds I-V and sofosbuvir, and pharmaceutical compositions thereof, described herein are effective in treating genotype 2 HCV infected patients, including genotype 2a, genotype 2b, genotype 2c and/or genotype 2d. In another embodiment, Compounds I-V and sofosbuvir, and pharmaceutical compositions thereof, as described herein are effective in treating genotype 3 HCV infected patients, including genotype 3a, genotype 3b, genotype 3c, genotype 3d, genotype 3e and/or genotype 3f. In another embodiment, Compounds I-V and sofosbuvir, and pharmaceutical compositions thereof, as described herein are effective in treating genotype 4 HCV infected patients, including genotype 4a, genotype 4b, genotype 4c, genotype 4d, genotype 4e, genotype 4f, genotype 4g, genotype 4h, genotype 4i and/or genotype 4j. In another embodiment, Compounds I-V and sofosbuvir, and pharmaceutical compositions thereof, as described herein are effective in treating genotype 5 HCV infected patients, including genotype 5a. In another embodiment, Compounds I-V and sofosbuvir, and pharmaceutical compositions thereof, as described herein are effective in treating genotype 6 HCV infected patients, including genotype 6a. In one embodiment, Compounds I-V and sofosbuvir, and pharmaceutical compositions thereof, as described herein are pangenotypic, meaning they are useful across all genotypes and drug resistant mutants thereof.

Each of Compounds I-V and sofosbuvir can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10. Typically, the compound will be administered in a dose from 0.01 milligrams to 2 grams. In one embodiment, the dose will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the compound may be administered once, twice or three times a day.

While it is possible for an active ingredient to be administered alone it may be preferable to present them as pharmaceutical formulations or pharmaceutical compositions as described below. The formulations, both for veterinary and for human use, of the disclosure comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulation for each of Compounds I-V and sofosbuvir can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For each of Compounds I-V and sofosbuvir, formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet, for each of Compounds I-V and sofosbuvir, can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Each of Compounds I-V and sofosbuvir can be administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this disclosure is that they are orally bioavailable and can be dosed orally.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as, for example, 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as, for example, propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this disclosure may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the disclosure include Tween® 60, Span® 80, cetostearyl alcohol, benzyl alcohol, myristyl alcohol, glyceryl monostearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as, for example, di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as, for example, white soft paraffin and/or liquid paraffin or other mineral oils are used.

Compound I has previously been described (see, for example, WO 2013/075029) and can be prepared by methods described therein. Compound II has previously been described (see, for example, U.S. Pat. No. 8,088,368) and can be prepared by methods described therein. Compound III has previously been described (see, for example, U.S. Pat. No. 8,513,298) and can be prepared by methods described therein. Compound IV has previously been described (see, for example, U.S. Pat. No. 8,178,491) and can be prepared by methods described therein. Compound V is a known compound.

Sofosbuvir has previously been described in U.S. Pat. No. 7,964,580 and U.S. Pat. Pub. Nos: 2010/0016251, 2010/0298257, 2011/0251152 and 2012/0107278.

In some embodiments, one or more of the compounds, Compounds I-V, and sofosbuvir are administered separately, either concurrently or sequentially. In some aspects, one or more of the compounds, Compounds I-V, are administered prior to sofosbuvir. In some aspects, sofosbuvir is administered prior to one or more of the compounds, Compounds I-V.

In one embodiment, the patient is human.

### 3. Additional Antiviral Agents

In some embodiments, the methods can further comprise the administration of another therapeutic agent for treating HCV and other conditions such as HIV infections. In one embodiment, non-limiting examples of suitable additional therapeutic agents include one or more interferons, ribavirin or its analogs, HCV NS3 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophillin inhibitors, HCV IRES inhibitors, pharmacokinetic enhancers, and other drugs or therapeutic agents for treating HCV.

More specifically, the additional therapeutic agent may be selected from the group consisting of:
1) interferons, e.g., pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha-2b XL, BLX-883 (Locteron), DA-3021, glycosylated interferon alpha-2b (AVI-005), PEG-Infergen, PEGylated interferon lambda-1 (PEGylated IL-29), and belerofon;
2) ribavirin and its analogs, e.g., ribavirin (Rebetol, Copegus), and taribavirin (Viramidine);
3) HCV NS3 protease inhibitors, e.g., boceprevir (SCH-503034, SCH-7), telaprevir (VX-950), TMC435350, BI-1335, BI-1230, MK-7009, VBY-376, VX-500, GS-9256, BMS-605339, PHX-1766, AS-101, YH-5258, YH5530, YH5531, ABT-450, ACH-1625, ITMN-191, MK5172, MK6325, and MK2748;
4) alpha-glucosidase 1 inhibitors, e.g., celgosivir (MX-3253), Miglitol, and UT-231B;
5) hepatoprotectants, e.g., emericasan (IDN-6556), ME-3738, GS-9450 (LB-84451), silibilin, and MitoQ;
6) nucleoside or nucleotide inhibitors of HCV NS5B polymerase, e.g., R1626, R7128 (R4048), IDX184, IDX-102, BCX-4678, valopicitabine (NM-283), MK-0608, and INX-189 (now BMS986094);
7) non-nucleoside inhibitors of HCV NS5B polymerase, e.g., PF-868554, VCH-759, VCH-916, JTK-652, MK-3281, VBY-708, VCH-222, A848837, ANA-598, GL60667, GL59728, A-63890, A-48773, A-48547, BC-2329, VCH-796 (nesbuvir), GSK625433, BILN-1941, XTL-2125, ABT-072, ABT-333, and PSI-7792;
8) HCV NS5A inhibitors, AZD-2836 (A-831), BMS-790052, ACH-3102, ACH-2928, MK8325, MK4882, MK8742, PSI-461, IDX719, ABT-267 and A-689;
9) TLR-7 agonists, e.g., imiquimod, 852A, GS-9524, ANA-773, ANA-975, AZD-8848 (DSP-3025), and SM-360320;
10) cyclophillin inhibitors, e.g., DEBIO-025, SCY-635, and NIM811;
11) HCV IRES inhibitors, e.g., MCI-067;
12) pharmacokinetic enhancers, e.g., BAS-100, SPI-452, PF-4194477, TMC-41629, GS-9350, GS-9585, and roxythromycin; and
13) other drugs for treating HCV, e.g., thymosin alpha 1 (Zadaxin), nitazoxanide (Alinea, NTZ), BIVN-401 (virostat), PYN-17 (altirex), KPE02003002, actilon (CPG-10101), GS-9525, KRN-7000, civacir, GI-5005, XTL-6865, BIT225, PTX-111, ITX2865, TT-033i, ANA 971, NOV-205, tarvacin, EHC-18, VGX-410C, EMZ-702, AVI 4065, BMS-650032, BMS-791325, Bavituximab, MDX-1106 (ONO-4538), Oglufanide, and VX-497 (merimepodib).

In another embodiment, the therapeutic agent used can be any agent having a therapeutic effect when used in combination with the other agents as described herein. For example, the therapeutic agent can be interferons, ribavirin analogs, NS3 protease inhibitors, NS5b polymerase inhibitors, alpha-glucosidase 1 inhibitors, hepatoprotectants, non-nucleoside inhibitors of HCV, and other drugs for treating HCV.

In certain embodiments, the additional therapeutic agent is selected from the group consisting of non-nucleoside inhibitors of HCV NS5B polymerase (ABT-072 and ABT-333), HCV NS5A inhibitors (ABT-267, ACH-3102 and ACH-2928) and HCV NS3 protease inhibitors (ABT-450 and ACH-1625).

In one embodiment, the additional therapeutic agent used in combination with the pharmaceutical compositions as described herein is a HCV NS3 protease inhibitor. Non-limiting examples include one or more compounds selected from the group consisting of:

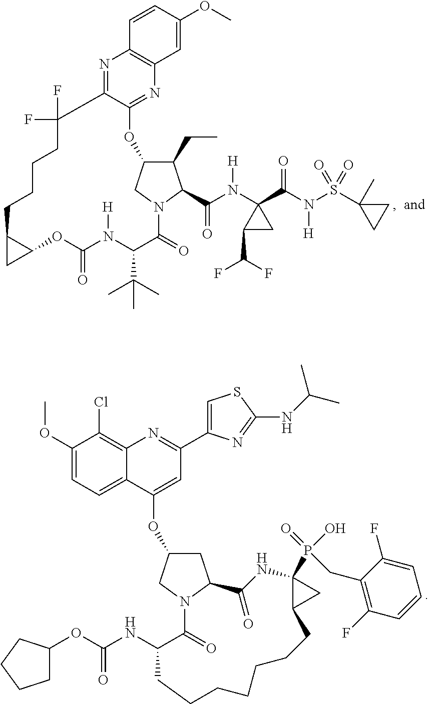 [SMILES: CC[C@@H]1[C@@H]2CN(C(=O)[C@H](C(C)(C)C)NC(=O)O[C@@H]3C[C@H]3CCCCC(F)(F)c3nc4ccc(OC)cc4nc3O2)[C@@H]1C(=O)N[C@]1(C(=O)NS(=O)(=O)C2(C)CC2)C[C@H]1C(F)F ; COc1ccc2c(O[C@@H]3C[C@H]4C(=O)N[C@]5(P(=O)(O)Cc6c(F)cccc6F)C[C@H]5CCCCCCC[C@H](NC(=O)OC5CCCC5)C(=O)N4C3)cc(-c3csc(NC(C)C)n3)nc2c1Cl]

In another embodiment, the additional therapeutic agent used in combination with the pharmaceutical compositions as described herein is a cyclophillin inhibitor, including for example, a cyclophilin inhibitor disclosed in WO 2013/185093. Non-limiting examples include one or more compounds selected from the group consisting of.

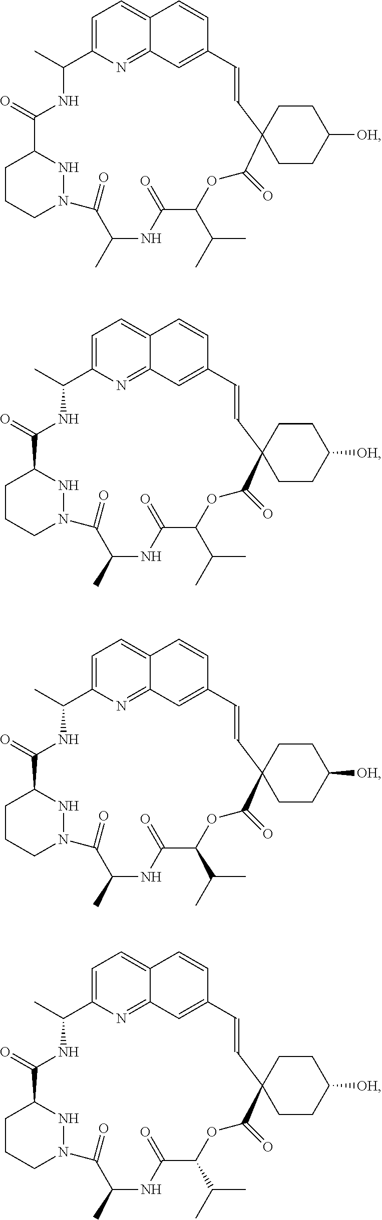 [SMILES: CC1NC(=O)C(C(C)C)OC(=O)C2(/C=C/c3ccc4ccc(nc4c3)C(C)NC(=O)C3CCCN(N3)C1=O)CCC(O)CC2 ; CC(C)C1OC(=O)[C@]2(/C=C/c3ccc4ccc(nc4c3)[C@@H](C)NC(=O)[C@@H]3CCCN(N3)C(=O)[C@H](C)NC1=O)CC[C@@H](O)CC2 ; CC(C)[C@@H]1OC(=O)[C@]2(/C=C/c3ccc4ccc(nc4c3)[C@@H](C)NC(=O)[C@@H]3CCCN(N3)C(=O)[C@H](C)NC1=O)CC[C@H](O)CC2 ; CC(C)[C@H]1OC(=O)[C@]2(/C=C/c3ccc4ccc(nc4c3)[C@@H](C)NC(=O)[C@@H]3CCCN(N3)C(=O)[C@H](C)NC1=O)CC[C@@H](O)CC2]

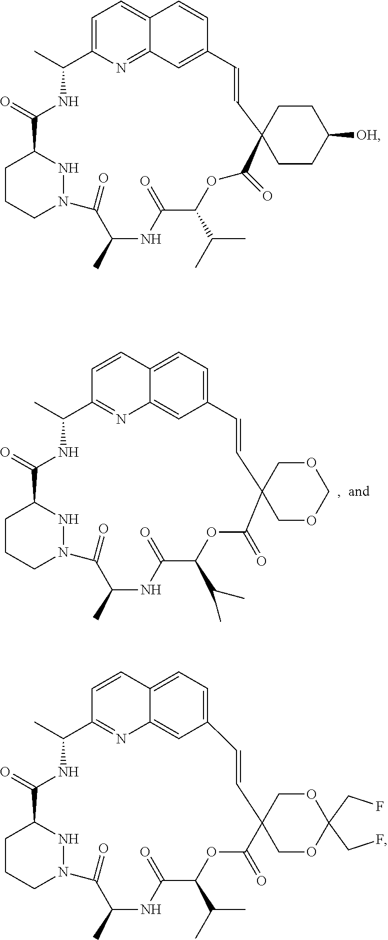 [SMILES: CC(C)[C@H]1OC(=O)[C@]2(/C=C/c3ccc4ccc(nc4c3)[C@@H](C)NC(=O)[C@@H]3CCCN(N3)C(=O)[C@H](C)NC1=O)CC[C@H](O)CC2 ; CC(C)[C@@H]1OC(=O)C2(/C=C/c3ccc4ccc(nc4c3)[C@@H](C)NC(=O)[C@@H]3CCCN(N3)C(=O)[C@H](C)NC1=O)COCOC2 ; CC(C)[C@@H]1OC(=O)C2(/C=C/c3ccc4ccc(nc4c3)[C@@H](C)NC(=O)[C@@H]3CCCN(N3)C(=O)[C@H](C)NC1=O)COC(CF)(CF)OC2]

and stereoisomers and mixtures of stereoisomers thereof.

In another embodiment, the additional therapeutic agent used in combination with the pharmaceutical compositions as described herein is a non-nucleoside inhibitor of HCV NS5B polymerase. A non-limiting example includes Compound X-3 as described below.

Examples of additional anti-HCV agents which can be combined with the compositions provided herein include, without limitation, the following:
A. interferons, for example, pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha XL, BLX-883 (Locteron), DA-3021, glycosylated interferon alpha-2b (AVI-005), PEG-Infergen, PEGylated interferon lambda (PEGylated IL-29), or belerofon, IFN alpha-2b XL, rIFN-alpha 2a, consensus IFN alpha, infergen, rebif, pegylated IFN-beta, oral interferon alpha, feron, reaferon, intermax alpha, r-IFN-beta, and infergen+actimmuneribavirin and ribavirin analogs, e.g., rebetol, copegus, VX-497, and viramidine (taribavirin);
B. NS5A inhibitors, for example, Compound II, Compound X-1 (described below), ABT-267, Compound X-2 (described below), JNJ-47910382, daclatasvir (BMS-790052), ABT-267, MK-8742, EDP-239, IDX-719, PPI-668, GSK-2336805, ACH-3102, A-831, A-689, AZD-2836 (A-831), AZD-7295 (A-689), and BMS-790052;
C. NS5B polymerase inhibitors, for example, Compound X-3 (described below), Compound X-4 (described below), ABT-333, Compound X-5 (described below), ABT-072, Compound X-6 (described below), tegobuvir (GS-9190), GS-9669, TMC647055, setrobuvir (ANA-598), filibuvir (PF-868554), VX-222, IDX-375, IDX-184, IDX-102, BI-207127, valopicitabine (NM-283), PSI-6130 (R1656), PSI-7851, BCX-4678, nesbuvir (HCV-796), BILB 1941, MK-0608, NM-107, R7128, VCH-759, GSK625433, XTL-2125, VCH-916, JTK-652, MK-3281, VBY-708, A848837, GL59728, A-63890, A-48773, A-48547, BC-2329, BMS-791325, and BILB-1941;
D. NS3 protease inhibitors, for example, Compound X-7, Compound X-8, Compound X-9, ABT-450, Compound X-10 (described below), simeprevir (TMC-435), boceprevir (SCH-503034), narlaprevir (SCH-900518), vaniprevir (MK-7009), MK-5172, danoprevir (ITMN-191), sovaprevir (ACH-1625), neceprevir (ACH-2684), Telaprevir (VX-950), VX-813, VX-500, faldaprevir (BI-201335), asunaprevir (BMS-650032), BMS-605339, VBY-376, PHX-1766, YH5531, BILN-2065, and BILN-2061;
E. alpha-glucosidase 1 inhibitors, for example, celgosivir (MX-3253), Miglitol, and UT-231B;
F. hepatoprotectants, e.g., IDN-6556, ME 3738, MitoQ, and LB-84451;
G. non-nucleoside inhibitors of HCV, e.g., benzimidazole derivatives, benzo-1,2,4-thiadiazine derivatives, and phenylalanine derivatives; and
H. other anti-HCV agents, e.g., zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975, XTL-6865, ANA 971, NOV-205, tarvacin, EHC-18, and NIM811.

Compound X-1 is an NS5A inhibitor and is represented by the following chemical structure:

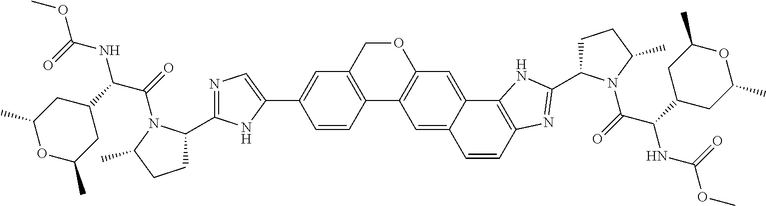

Compound X-2 is an NS5A inhibitor and is represented by the following chemical structure:

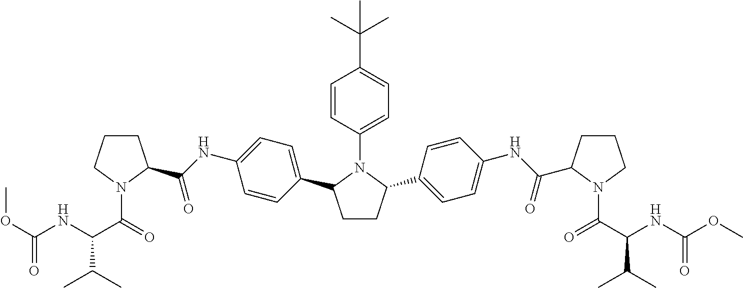

(see, e.g., U.S. Publication No. 2013/0102525 and references therein).

Compound X-3 is an NS5B Thumb II polymerase inhibitor and is represented by the following chemical structure:

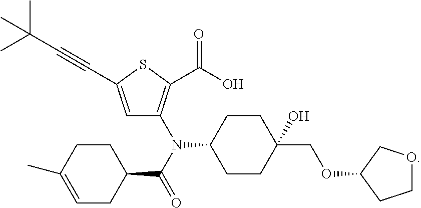 [SMILES: CC1=CC[C@H](C(=O)N(c2cc(C#CC(C)(C)C)sc2C(=O)O)[C@H]2CC[C@](O)(CO[C@H]3CCOC3)CC2)CC1]

Compound X-4 is a nucleotide inhibitor prodrug designed to inhibit replication of viral RNA by the HCV NS5B polymerase, and is represented by the following chemical structure:

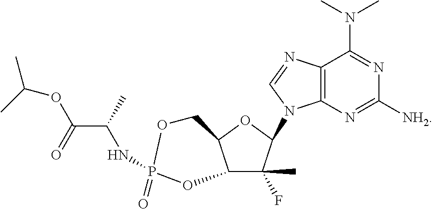 [SMILES: CC(C)OC(=O)[C@H](C)N[P@]1(=O)OC[C@H]2O[C@@H](n3cnc4c(N(C)C)nc(N)nc43)[C@](C)(F)[C@@H]2O1]

Compound X-5 is an HCV polymerase inhibitor and is represented by the following structure:

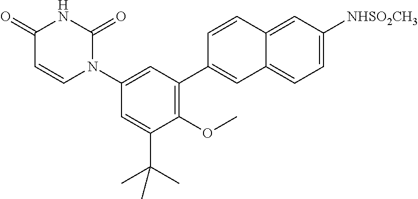

(see, e.g., U.S. Publication No. 2013/0102525 and references therein).

Compound X-6 is an HCV polymerase inhibitor and is represented by the following structure:

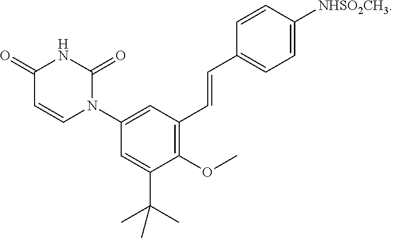

(see, e.g., U.S. Publication No. 2013/0102525 and references therein).

Compound X-7 is an HCV protease inhibitor and is represented by the following chemical structure:

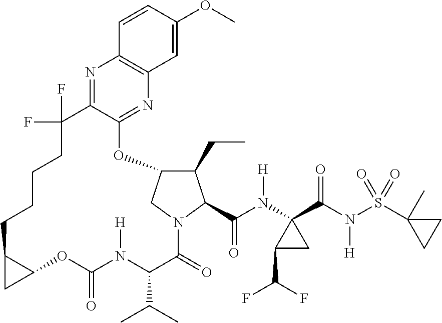 [SMILES: CC[C@@H]1[C@@H]2CN(C(=O)[C@H](C(C)C)NC(=O)O[C@@H]3C[C@H]3CCCCC(F)(F)c3nc4ccc(OC)cc4nc3O2)[C@@H]1C(=O)N[C@]1(C(=O)NS(=O)(=O)C2(C)CC2)C[C@H]1C(F)F]

(see e.g., U.S. Publication No. 2014/0017198 and references therein).

Compound X-8 is an HCV protease inhibitor and is represented by the following chemical structure:

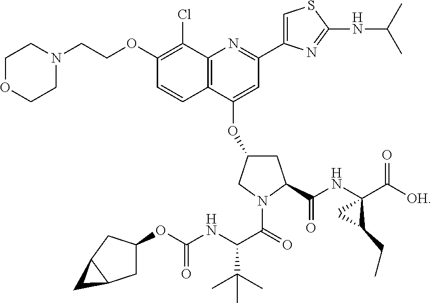

(see e.g., U.S. Pat. No. 8,178,491 and references therein).

Compound X-9 is an HCV protease inhibitor and is represented by the following chemical structure:

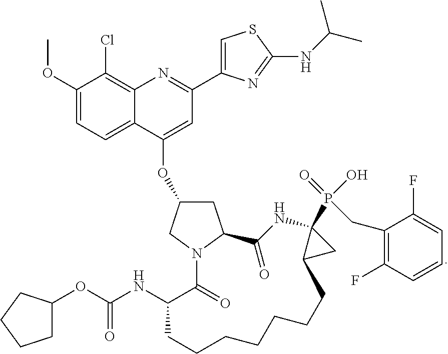 [SMILES: COc1ccc2c(O[C@@H]3C[C@H]4C(=O)N[C@]5(P(=O)(O)Cc6c(F)cccc6F)C[C@H]5CCCCCCC[C@H](NC(=O)OC5CCCC5)C(=O)N4C3)cc(-c3csc(NC(C)C)n3)nc2c1Cl]

Compound X-10 is an HCV protease inhibitor and is represented by the following chemical structure:

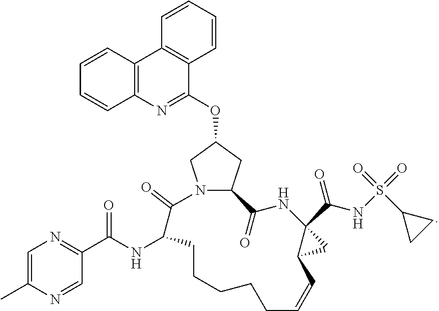 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

(see, e.g., U.S. Publication No. 2013/0102525 and references therein).

In another embodiment, the present application provides for a method of treating hepatitis C in a human patient in need thereof comprising administering to the patient an effective amount of sofosbuvir, an effective amount of a compound selected from the group consisting of Compound I, Compound II, Compound III, Compound IV, and Compound V or a combination thereof, and an additional therapeutic selected from the group consisting of pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, r-IFN-beta, infergen+actimmune, IFN-omega with DUROS, albuferon, rebetol, copegus, levovirin, VX-497, viramidine (taribavirin), A-831, A-689, NM-283, valopicitabine, R1626, PSI-6130 (R1656), HCV-796, BILB 1941, MK-0608, NM-107, R7128, VCH-759, PF-868554, GSK625433, XTL-2125, SCH-503034 (SCH-7), VX-950 (Telaprevir), ITMN-191, and BILN-2065, MX-3253 (celgosivir), UT-231B, IDN-6556, ME 3738, MitoQ, and LB-84451, benzimidazole derivatives, benzo-1,2,4-thiadiazine derivatives, and phenylalanine derivatives, zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811 and a pharmaceutically acceptable carrier or excipient.

The additional therapeutic agent may be one that treats other conditions such as HIV infections. Accordingly, the additional therapeutic agent may be a compound useful in treating HIV, for example HIV protease inhibiting compounds, non-nucleoside inhibitors of HIV reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, interferons, ribavirin analogs, NS3 protease inhibitors, NS5b polymerase inhibitors, alpha-glucosidase 1 inhibitors, hepatoprotectants, non-nucleoside inhibitors of HCV, and other drugs for treating HCV.

More specifically, the additional therapeutic agent may be selected from the group consisting of
1) HIV protease inhibitors, e.g., amprenavir, atazanavir, fosamprenavir, indinavir, lopinavir, ritonavir, lopinavir+ritonavir, nelfinavir, saquinavir, tipranavir, brecanavir, darunavir, TMC-126, TMC-114, mozenavir (DMP-450), JE-2147 (AG1776), AG1859, DG35, L-756423, R00334649, KNI-272, DPC-681, DPC-684, and GW640385X, DG17, PPL-100,
2) a HIV non-nucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirine), efavirenz, BILR 355 BS, VRX 840773, UK-453,061, RDEA806,
3) a HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (±-FTC), D-d4FC, emtricitabine, phosphazide, fozivudine tidoxil, fosalvudine tidoxil, apricitibine (AVX754), amdoxovir, KP-1461, abacavir+lamivudine, abacavir+lamivudine+zidovudine, zidovudine+lamivudine,
4) a HIV nucleotide inhibitor of reverse transcriptase, e.g., tenofovir, tenofovir disoproxil fumarate+emtricitabine, tenofovir disoproxil fumarate+emtricitabine+efavirenz, and adefovir,
5) a HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-707035, MK-2048, BA-011, BMS-538158, GSK364735C,
6) a gp4l inhibitor, e.g., enfuvirtide, sifuvirtide, FB006M, TRI-1144, SPC3, DES6, Locus gp41, CovX, and REP 9,
7) a CXCR4 inhibitor, e.g., AMD-070,
8) an entry inhibitor, e.g., SP01A, TNX-355,
9) a gp120 inhibitor, e.g., BMS-488043 and BlockAide/CR,
10) a G6PD and NADH-oxidase inhibitor, e.g., immunitin,
11) a CCR5 inhibitor, e.g., aplaviroc, vicriviroc, INCB9471, PRO-140, INCB15050, PF-232798, CCR5mAb004, and maraviroc,
12) a CCR5 inhibitor, e.g., aplaviroc, vicriviroc, INCB9471, PRO-140, INCB15050, PF-232798, CCR5mAb004, and maraviroc,
13) an interferon, e.g., pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, r-IFN-beta, infergen+actimmune, IFN-omega with DUROS, and albuferon,
14) ribavirin analogs, e.g., rebetol, copegus, levovirin, VX-497, and viramidine (taribavirin)
15) NS5a inhibitors, e.g., A-831, A-689, ABT-267 and BMS-790052,
16) NS5b polymerase inhibitors, e.g., NM-283, valopicitabine, R1626, PSI-6130 (R1656), HCV-796, BILB 1941, MK-0608, NM-107, R7128, VCH-759, PF-868554, GSK625433, and XTL-2125,
17) NS3 protease inhibitors, e.g., SCH-503034 (SCH-7), VX-950 (Telaprevir), ITMN-191, ABT-450 and BILN-2065,
18) alpha-glucosidase 1 inhibitors, e.g., MX-3253 (celgosivir) and UT-231B,
19) hepatoprotectants, e.g., IDN-6556, ME 3738, MitoQ, and LB-84451,
20) non-nucleoside inhibitors of HCV, e.g., benzimidazole derivatives, ABT-072, ABT-333, benzo-1,2,4-thiadiazine derivatives, and phenylalanine derivatives,
21) other drugs for treating Hepatitis C, e.g., zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811,
22) pharmacokinetic enhancers, e.g., BAS-100 and SPI452, 20) RNAse H inhibitors, e.g., ODN-93 and ODN-112, and
23) other anti-HIV agents, e.g., VGV-1, PA-457 (bevirimat), ampligen, HRG214, cytolin, polymun, VGX-410, KD247, AMZ 0026, CYT 99007, A-221 HIV, BAY 50-4798, MDX010 (iplimumab), PBS119, ALG889, and PA-1050040.

It is contemplated that the additional therapeutic agent will be administered in a manner that is known in the art and the dosage may be selected by someone of skill in the art. For example, the additional agent may be administered in a dose from about 0.01 milligrams to about 2 grams per day.

### 3. Pharmaceutical Compositions

The pharmaceutical compositions of the disclosure provide for an effective amount of Compound I, Compound II, Compound III, Compound IV, Compound V, or a combination thereof and an effective amount of sofosbuvir. In some embodiments, the amount of sofosbuvir is 350 mg daily or less.

Pharmaceutical formulations according to the present disclosure comprise Compounds I-V and sofosbuvir together with one or more pharmaceutically acceptable carriers or excipients and optionally other therapeutic agents. Pharmaceutical formulations containing the active ingredient (i.e. Compounds I-V and sofosbuvir) may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as, for example, gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as, for example, 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as, for example, compounds heretofore used in the treatment or prophylaxis of conditions associated with HCV activity.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this disclosure may include other agents conventional in the art having regard to the type of formulation in question, for example those suitable for oral administration may include flavoring agents.

The disclosure further provides veterinary compositions comprising an effective amount of Compound I, Compound II, Compound III, Compound IV, Compound V, or a combination thereof and an effective amount of sofosbuvir together with a veterinary carrier therefore.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds I-V and sofosbuvir can also be formulated to provide controlled release of the active ingredient to allow less frequent dosing or to improve the pharmacokinetic or toxicity profile of the active ingredient. Accordingly, the disclosure also provides compositions comprising an effective amount of Compound I, Compound II, Compound III, Compound IV, Compound V, or a combination thereof and an effective amount of sofosbuvir formulated for sustained or controlled release.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies.

### EXAMPLES

In the following examples and throughout this disclosure, abbreviations as used herein have respective meanings as follows:

| | |
|---|---|
| λ_{z} | Elimination rate constant |
| AE | Adverse event |
| AUC | Area under the curve |
| % AUCₑₓₜᵣₐₚ | Percentage of extrapolated area under the |
| | curve |
| AUC_{inf} | Area under the curve from time zero to |
| | infinity |
| AUCₗₐₛₜ | Area under the curve from time zero to the |
| | time of last quantifiable concentration |
| BSA | Bovine serum albumin |
| CI | Confidence Intervals |
| CL/F | Oral clearance |
| cm | Centimeter |
| Cₘₐₓ | Maximum concentration |
| CV | Covariance |
| DCM | Dichloromethane |
| ECG | Electrocardiogram |
| EDTA | Ethylenediaminetetraacetic acid |
| GERD | Gastroesophageal reflux disease |
| GMR | Geometric mean rise |
| h or hr | Hour |
| HBSSg | Hanks buffer |
| HCV | Hepatitis C virus |
| HIV | Human immunodeficiency virus |
| IFN | Interferon |
| kg | Kilogram |
| LC/MS/MS | Liquid chromatography-tandem mass |
| | spectrometry |
| MAD | Multiple ascending dose |
| m | Meter |
| M | Molar |
| Me | Methyl |
| mg | Milligram |
| min | Minute |
| mL | Milliliter |
| ng | Nanogram |
| PEG | Polyethylene glycol |
| PK | Pharmacokinetics |
| RNA | Ribonucleic acid |
| s | second |
| SVR | Sustained virologic response |
| T_{1/2} | Apparent terminal half-life |
| Tₗₐₛₜ | Last time point at which quantifiable drug |
| | concentration can be measured |
| Tₘₐₓ | Time at Cₘₐₓ |
| TEER | Transendothelial electrical resistance |
| μm | Micrometer |
| μM | Micromolar |

### Example 1

### Pharmacogenic Study of the Combination of Sofosbuvir with Compound I

This example studied the pharmacokinetics (PK) of the combination of Compound I and sofosbuvir (SOF), along with two metabolites of SOF, Metabolite I and II, in healthy volunteers, as part of a Phase I clinical study for Compound I.

This was a randomized, open label, single-center, multiple-dose study that enrolled Healthy males and non-pregnant, non-lactating females from 18 to 45 years old. The duration of the study was up to 38 days, and duration of the treatment was 28 days. Eligible subjects were an approximately even distribution of males and nonpregnant, nonlactating females, with a body mass index (BMI) from 19 to 30 kg/m², ECG without clinically significant abnormalities, estimated creatinine clearance (calculated using the Cockcroft-Gault equation)≧80 mL/min, no significant medical history and in good general health as determined by the investigator at screening evaluation performed no more than 28 days prior to the scheduled first dose of study drug.

Additional specific exclusion criteria included, (1) use of over the counter (OTC) or prescribed medications that affect gastric pH (i.e., antacids, H2 receptor antagonists (H2RAs), and/or proton-pump inhibitors (PPIs)) 28 days prior to baseline and (2) history of severe peptic ulcer disease, GERD, or other stomach acid diseases requiring prolonged (>6 months) medication or surgical therapy to modify gastric pH.

A single dose of Compound I, 100 mg (2×50 mg active tablets), was administered alone, or simultaneously with a single dose of SOF, under fasted conditions.

Safety were assessed during the study by clinical laboratory tests, physical examinations including vital signs and electrocardiograms (ECGs) at various time points during the study, and by documentation of adverse events and concomitant medications throughout the study.

Pharmacokinetic: The following PK parameters were calculated: Cₘₐₓ, Tₘₐₓ, Tₗₐₛₜ, λ_{z}, AUC_{inf}, AUCₗₐₛₜ, % AUCₑₓₜᵣₐₚ, CL/F, and T_{1/2}.

Plasma concentrations and pharmacokinetic parameters for the compounds were listed and summarized using descriptive statistics by treatment. In addition, an analysis of variance (ANOVA) using a mixed-effects model with treatment, period, sequence as fixed effects and subject within sequence as a random effect were fitted to the natural logarithmic transformation of PK parameters (AUC_{inf}, AUCₗₐₛₜ, and Cₘₐₓ).

The 90% confidence intervals were constructed for the ratio of geometric least-squares means of primary PK parameters for each of the test treatments (as shown in Table 1).

Safety and AEs: Safety data were listed by subject and summarized by treatment. Treatment-emergent AEs will be summarized by treatment, system organ class and preferred term using the most current version of the Medical Dictionary for Regulatory Activities (MedDRA). All AEs and all treatment-related AEs were listed by subject. The frequency of subjects who experienced AEs were summarized overall by treatment. AEs were also summarized by relationship to study drug and severity. In addition, a list of AEs leading to discontinuation of study drug or study prematurely were provided.

Listings of individual subject laboratory results were provided. Selected laboratory data were summarized by treatment at scheduled visits and for the corresponding change from Baseline. The incidence of treatment-emergent graded laboratory abnormalities was summarized by treatment.

The obtained pharmacokinetic results are presented in Table 1 below.

TABLE 1
Pharmacokinetic data for Compound I (Comp I) and sofosbuvir (SOF),
Metabolite I or II, both of which are metabolites of SOF

| | | |
|---|---|---|
| Mean (% CV) | SOF + Comp I | % GMR (90% CI) |

SOF alone

SOF (n = 18)

| | | | | | | |
|---|---|---|---|---|---|---|
| AUC_{inf} (ng · hr/mL) | 1200 | (26.7) | 2860 | (26.3) | 238 | (214, 264) |
| AUCₗₐₛₜ | 1200 | (26.8) | 2850 | (26.4) | 238 | (215, 264) |
| (ng · hr/mL) | | | | | | |
| Cₘₐₓ (ng/mL) | 964 | (47.5) | 1670 | (30.2) | 181 | (149, 219) |

Metabolite I (n = 18)

| | | | | | | |
|---|---|---|---|---|---|---|
| AUC_{inf} (ng · hr/mL) | 1990 | (26.2) | 3540 | (21.4) | 179 | (165, 195) |
| AUCₗₐₛₜ | 1930 | (26.8) | 3480 | (21.7) | 182 | (168, 199) |
| (ng · hr/mL) | | | | | | |
| Cₘₐₓ (ng/mL) | 476 | (30.7) | 751 | (19.3) | 162 | (145, 180) |

Metabolite II (n = 18)

| | | | | | | |
|---|---|---|---|---|---|---|
| AUC_{inf} (ng · hr/mL) | 12200 | (21.7) | 14200 | (21.4) | 117 | (111, 122) |
| AUCₗₐₛₜ (ng · hr/mL) | 11400 | (19.9) | 12900 | (20.5) | 113 | (108, 118) |
| Cₘₐₓ (ng/mL) | 1110 | (23.9) | 703 | (16.9) | 64.2 | (58.4, 70.4) |

Comp I alone

Compound I (n = 18)

| | | | | | | |
|---|---|---|---|---|---|---|
| AUCₜₐᵤ (ng · hr/mL) | 7690 | (30.5) | 8600 | (30.3) | 112 | (108, 116) |
| Cₘₐₓ (ng/mL) | 963 | (24.1) | 1020 | (24.4) | 106 | (102, 110) |
| Cₜₐᵤ (ng/mL) | 113 | (44.0) | 135 | (46.6) | 118 | (112, 124) |

GMR: Geometric mean rise;
Data presented as 3 significant figures.

As shown in Table 1, SOF AUC and Cₘₐₓ were increased by ˜2.4-fold and ˜1.8-fold, respectively by Compound I. A similar increase in Metabolite I systemic exposure (˜1.6-1.8-fold) was observed with Metabolite I. By contrast, for Metabolite II, an approximately 35% lower C. was observed upon co-administration with Compound I with no apparent change in AUC.

### Example 2

### Compound I Inhibits P-Gp but not Other Transporters

Based on the results of Example 1, it was contemplated that Compound I increased the exposure of sofosbuvir by inhibiting the activities of one or more drug transporters. This example carried out in vitro experiments to test whether Compound I inhibited any of the potentially relevant drug transporters, OAT1B1, OAT1B3, BCRP and P-gp.

Compound I exhibited marked inhibition on P-gp, but not on other tested transporters. As sofosbuvir is a known substrate for P-gp, this example demonstrates that co-administration of Compound I increases the exposure of sofosbuvir through inhibition of P-gp.

### Example 3

### Compounds I-V Increase the Absorption of Sofosbuvir by Inhibiting its Efflux

This example provides an assessment of the effect of Compounds I-V on the bidirectional permeability of sofosbuvir in vitro using a human colon carcinoma cell line (caco-2). Monolayers of caco-2 cells seeded in 12 well trans-well plates were found to meet acceptance criteria. TEER value, atenolol and lucifer yellow permeability indicated appropriate membrane integrity. Propranolol had high permeability. Transport was observed for a control transport substrate (digoxin).

Sofosbuvir showed low permeability and efflux (efflux ratio=43.6) through caco-2 cells when incubated alone at 10 μM. Coincubation with each of Compounds I-V affected the permeability of sofosbuvir causing increases in the apical to basolateral permeability, decreases in basolateral to apical permeability and reducing the efflux ratio. More specifically, Compound I had moderate effects of almost 4-fold increase. Compounds II, III, and V had moderate effect of almost 2-fold increase. Compound IV completely inhibited efflux transport. These results indicates that each of Compounds I-V can increase the absorption of sofosbuvir from the gastrointestinal tract due to its inhibition of sofosbuvir efflux.

### Methods

### Assay System

Caco-2 human intestinal epithelial cell monolayers cultured for twenty-one to twenty-eight days were plated on 12 well Transwell® dual chamber plates.

### Assay Conditions for Bidirectional Permeability Assessment

Assays were run in duplicate (n=2) in one to two separate experiments. Assay buffer at pH 7.4 was placed in both chambers. Hanks buffer with 1% BSA was used in receiver compartment.

Treatments with each of Compounds I-V were pre-incubated with each of Compounds I-V for 30 minutes prior to assay onset. Treatments without Compounds I-V were pre-incubated with blank HBSSg for 30 minutes prior to assay onset.

After the 30-min pre-incubation with each of Compounds I-V or buffer only, there was a 5-minute preincubation with the dosing solution followed by fresh donor and receiver buffer addition at which point time was recorded as time 0 (start of the experiment). Apical side was dosed for apical-to-basolateral (A to B) assessment. Basolateral side was dosed for basolateral-to-apical (B to A) assessment. Donor side was sampled at time points of 0 and 120 minutes.

Receiver side was sampled at time points: 60 and 120 minutes. Samples were analyzed for Compounds I-V concentrations by LC/MS/MS.

The apparent permeability, Pₐₚₚ, and % recovery were calculated as follows:

Pₐₚₚ=(dR/dt)×Vᵣ/(A×D₀)

% Recovery=100×((Vᵣ×R₁₂₀)+(V_{d}×D₁₂₀))/(Vd×D₀)

Where, dR/dt is the slope of the cumulative concentration in the receiver compartment versus time in μM/s based on receiver concentrations measured at 60 and 120 minutes. Vᵣ and V_{d} are the volumes in the receiver and donor compartment in cm³, respectively. A is the area of the cell monolayer (1.12 cm²). D₀ and D₁₂₀ are the measured donor concentrations at the beginning and end of the experiment, respectively. R₁₂₀ is the receiver concentration at the end of the experiment (120 minutes).

### Results

The cells used in these assays passed all the acceptance criteria. Each of Compounds I-V was incubated near its aqueous solubility limits to reflect possible concentrations obtained in the intestinal tract.

As summarized in Table 2, sofosbuvir showed low apical to basolateral permeability (Pₐₚₚ of 0.25×10⁻⁶ cm/s) with efflux (efflux ratio=43.6) in caco-2 cells when incubated alone at 10 μM. Coincubation with each of Compounds I-V affected the permeability of sofosbuvir causing increases in the apical to basolateral permeability, decreases in basolateral to apical permeability and reducing the efflux ratio. Almost 4-fold increase was observed with Compound I, an NS5A inhibitor. Compounds II, III, and V had moderate effect of almost 2-fold increase. Compound IV completely inhibited efflux transport.

TABLE 2
Effect of Compounds I-V on the Bi-Directional Permeability
of sofosbuvir in Monolayers of Caco-2 Cells

| | | Pₐₚₚ (10⁻⁶ cm/s) | | | Efflux |
|---|---|---|---|---|---|
| Direction | Inhibitor | Repeat 1 | Repat 2 | Average | Ratio |
| Forward | Compound I | 0.73 | 0.6 | 0.66 | 11.2 |
| Reverse | (1 μM) | 7.22 | 7.56 | 7.39 | |
| Forward | Compound II | 0.21 | 0.49 | 0.35 | 22.9 |
| Reverse | (1 μM) | 7.43 | 8.78 | 8.1 | |
| Forward | Compound III | 0.31 | 0.28 | 0.3 | 23.4 |
| Reverse | (100 μM) | 7.05 | 6.82 | 6.93 | |
| Forward | Compound IV | 0.78 | 1.41 | 1.09 | 1.1 |
| Reverse | (30 μM) | 1.08 | 1.25 | 1.17 | |
| Forward | Compound V | 0.46 | 0.92 | 11.1 | 16.1 |
| Reverse | (10 μM) | 10.5 | 11.7 | 1.09 | |

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method for treating a patient infected with hepatitis C virus comprising administering to the patient an effective amount of a compound selected from the group consisting of Compound I, Compound II, Compound III, Compound IV, and Compound V having the formulae: 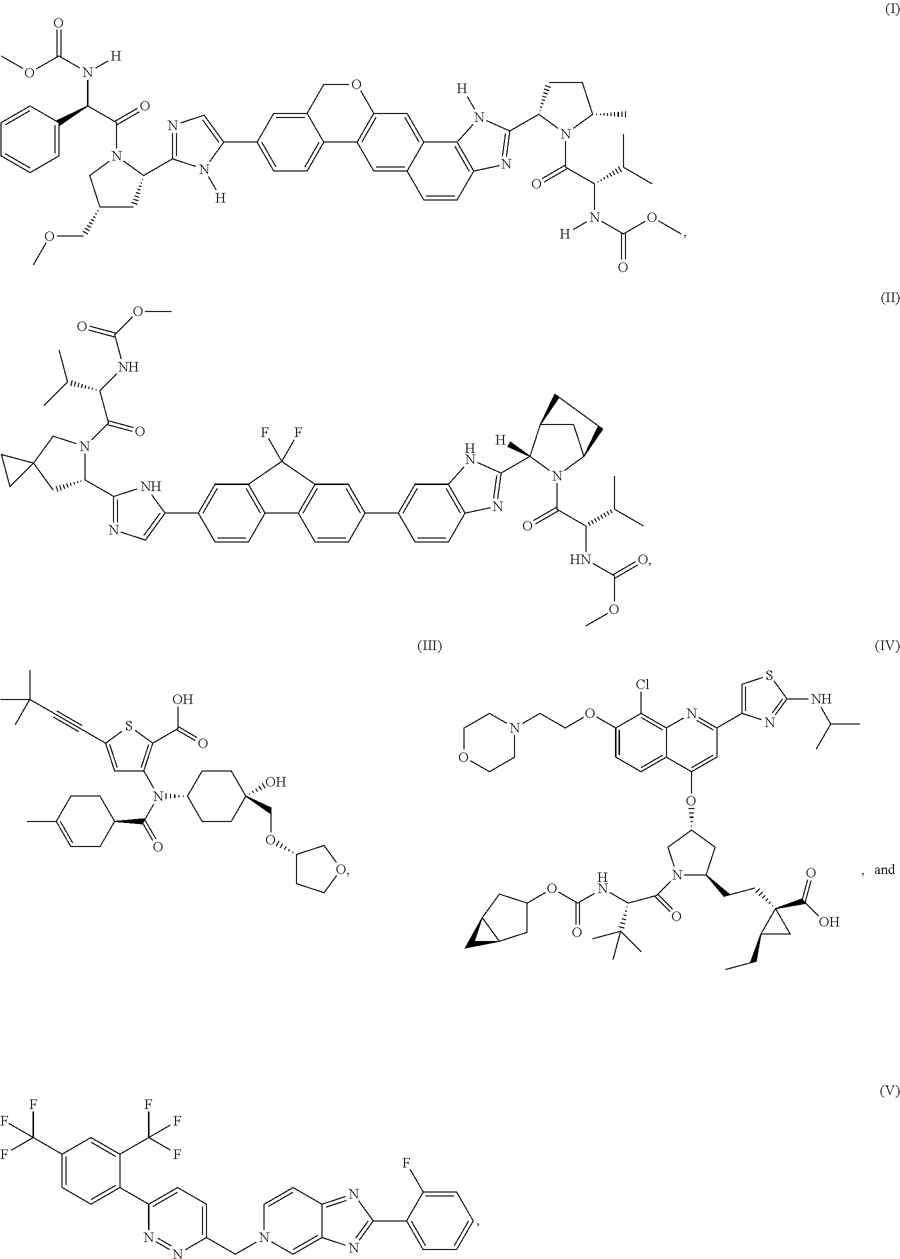
or a combination thereof, and
an effective amount of sofosbuvir having the formula 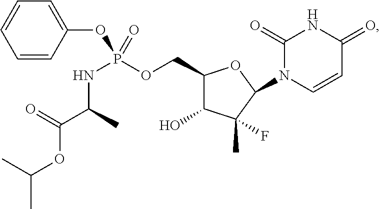 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
wherein the amount of sofosbuvir is about 350 mg daily or less.

2. The method of claim 1, wherein the amount of sofosbuvir is about 300 mg daily or less.

3. The method of claim 1, wherein the amount of sofosbuvir is from about 200 mg daily to about 350 mg daily.

4. The method of claim 1, wherein the amount of sofosbuvir is from about 250 mg daily to about 350 mg daily.

5. The method of claim 1, wherein the administration is carried out for a period from about 2 weeks to about 6 weeks.

6. The method of claim 5, wherein the administration is carried out for a period from about 4 weeks to about 6 weeks.

7. A pharmaceutical composition comprising a compound selected from the group consisting of Compound I, Compound II, Compound III, Compound IV, and Compound V having the formulae: 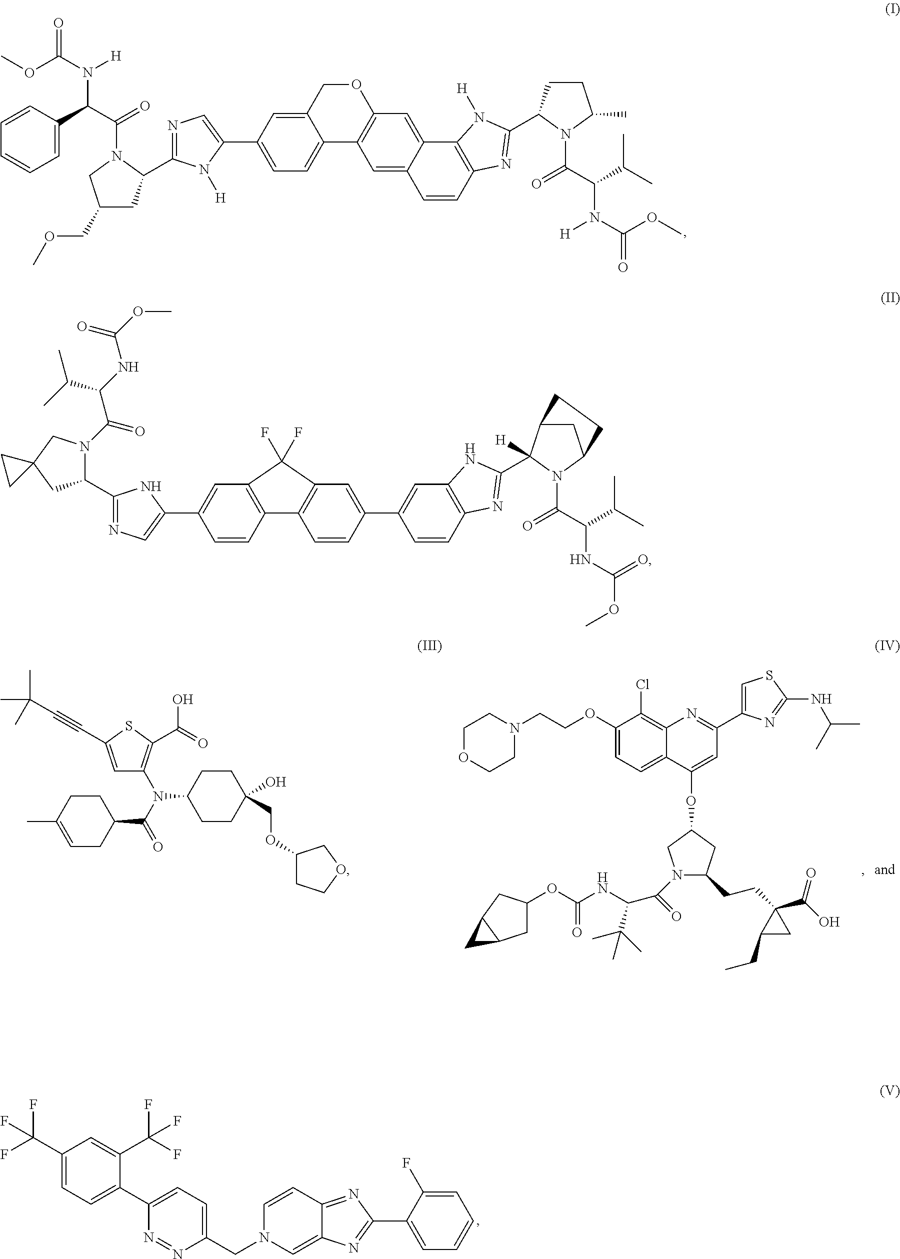
or a combination thereof, and
an effective amount of sofosbuvir having the formula 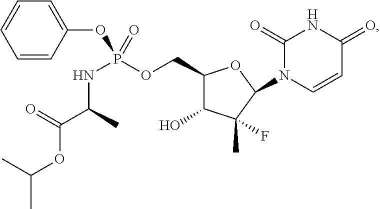 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
wherein the amount of sofosbuvir is about 350 mg or less.

8. The method of claim 1, wherein the pharmaceutical composition is administered once daily for about 8 weeks or less and wherein the hepatitis C virus is genotype 1, 2, 3, 4, 5, or 6.

9. The method of claim 1, wherein the pharmaceutical composition is administered once daily for about 6 weeks or less and wherein the hepatitis C virus is genotype 1, 2, 3, 4, 5, or 6.
